SEQUENCE LISTING (1 sequence)

SEQ ID NO: 1 - DNA, 60 bp, Artificial Sequence
  Designed 60-mer DNA molecule for testing diffusiophoresis sensing
agagagagagagagagagagagagagagagagagagagagagagagagagagagagagag

(19) United States
(12) Patent Application Publication
(10) Pub. No.: US 2022/0365064 A1
(43) Pub. Date: Nov. 17, 2022

(54) ANALYZING APPARATUS AND METHOD USING A PORE DEVICE

(71) Applicant: THE UNIVERSITY OF TOKYO, Tokyo (JP)

(72) Inventors: Wei-Lun HSU, Tokyo (JP); Hirofumi DAIGUJI, Tokyo (JP)

(21) Appl. No.: 17/536,655

(22) Filed: Nov. 29, 2021

Related U.S. Application Data
(63) Continuation of application No. PCT/JP2020/021113, filed on May 28, 2020.
(60) Provisional application No. 62/853,471, filed on May 28, 2019.

(51) Int. Cl. G01N 33/487; C12Q 1/6869

(52) U.S. Cl. CPC G01N 33/48721; C12Q 1/6869

(57) ABSTRACT

An analyzing apparatus for molecules is provided. A pore device includes a cation selective nanopore, and a first chamber and a second chamber which are separated by the cation selective nanopore. In an initial state, the first chamber includes molecules to be analyzed, and the second chamber has higher salt concentration than the first chamber. A current sensor measures an ionic current flowing through a first electrode and a second electrode provided in the first chamber and the second chamber.

### CROSS REFERENCE TO PRIOR APPLICATIONS

This application is a continuation under 35 U.S.C. § 120 of PCT/JP2020/021113, filed May 28, 2020, which is incorporated herein reference and which claimed priority to U.S. Provisional Application No. 62/853,471, filed May 28, 2019. The present application likewise claims priority to U.S. Provisional Application No. 62/853,471, filed May 28, 2019, the entire content of which is also incorporated herein by reference.

### INCORPORATION-BY-REFERENCE OF MATERIAL SUBMITTED ELECTRONICALLY

Incorporated by reference in its entirety herein is a computer-readable nucleotide/amino acid sequence listing submitted electronically in ASCII format and identified as follows: 620 byte ASCII (Text) file named “PRM0724USC_ST25” created Jul. 29, 2022.

### BACKGROUND

### 1. Technical Field

The present disclosure relates to an analyzing apparatus and method using a pore device.

### 2. Description of the Related Art

In human bodies, massive genomic information is recorded in our DNAs playing as a critical role in the development of next generation of diagnosis systems toward precision medicine technologies. Biological nanopore-based DNA sequencers embedded in a lipid bilayer have emerged as a rapid and inexpensive alternative to conventional DNA sequencing methods. The success of the fast DNA sequencing has stimulated the industry in precision medicine and quick diagnosis. In the past decade, the research in the field has bloomed unprecedentedly attributed to the enormous market potential for next generation of health care.

However, DNA sequencing using biological nanopores has struggled with the disadvantages of weak mechanical strength and high chemical sensitivity not only reducing the sensing accuracy but also increase the cost in replacing the nanopore membranes after each measurement (Non-patent document 1). In principal, artificial solid-state nanopores possess the advantages of mechanical strength, flexible geometry and stable chemical properties over the biological nanopores, and therefore are more favorable for biomolecule detections. However, since the concept was proposed in the beginning of this century, resistive pulse sensing using solid-state nanopores has been confronted by both spatial and temporal resolution limitations hindering them from practical sequencing applications.

Compared with the gap between DNA base pairs, being merely 0.34 nm, the thickness of the thinnest silicon nitride membranes is in the order of several nanometers. In this regard, two-dimensional materials nanopores have been recently adopted to enhance spatial resolution whose thicknesses coincide with the distance between each nucleotide (e.g. the thickness of a monolayer molybdenum disulfide is 0.65 nm) (Non-patent document 2). Nevertheless, although these ultrathin nanopores conceptually promise single nucleotide resolution, there exists a tremendous temporal resolution issue and thus no directly DNA sequencing results have been achieved due to the excessively fast translocation of the molecules through the nanopore (Non-patent document 3). Another challenging issue could be concurrent Joule heating effects when applying a significant electric potential difference over a short distance, resulting in high sensing noise and superheating effects in the nanopore which may alter the physical properties the DNA molecules (Non-patent document 4).

To circumvent these obstacles, we invent a method based on diffusiophoretic transport of DNA molecules through a nanopore when a salt concentration difference is applied across a nanopore without the application of an electric potential difference, preventing the issues due to the external electric field.

### CITATION LIST

### (1) Non Patent Literature

NPL1: D. Branton et al., Nat. Biotechnol., 26-10 (2008), 1146.

NPL2: J. Feng et al., Nat. Nanotechnol., 10 (2015), 1070.

NPL3: K. Lee et al., Adv. Mater., 30 (2018), 1704680.

NPL4: E. V. Levine et al., Phys. Rev. E, 93 (2016), 013124.

NPL5: Z. Gu et al., Sci. Bull., 62 (2017), 1245.

NPL6: C. R. Dean et al., Nat. Nanotechnol., 5 (2010), 722.

NPL7 J.-P. Hsu et al., Langmuir, 25-3 (2009), 1772.

### SUMMARY

The present disclosure has been made in view of the aforementioned situation.

A summary of several example embodiments of the disclosure follows. This summary is provided for the convenience of the reader to provide a basic understanding of such embodiments and does not wholly define the breadth of the disclosure. This summary is not an extensive overview of all contemplated embodiments, and is intended to neither identify key or critical elements of all embodiments nor to delineate the scope of any or all aspects. Its sole purpose is to present some concepts of one or more embodiments in a simplified form as a prelude to the more detailed description that is presented later.

In one embodiment, an apparatus and/or method of nanopore molecule sensing is provided. The apparatus/method uses ionic current generated as an electrolyte concentration gradient is applied across an ion selective nanopore.

In one embodiment, this salinity gradient method may be combined with a two-dimensional nanopore to achieve high spatial and temporal resolutions for various molecule sequencing and analysis applications.

It is to be noted that any arbitrary combination or rearrangement of the above-described structural components and so forth is effective as and encompassed by the present embodiments. Moreover, this summary of the invention does not necessarily describe all necessary features so that the invention may also be a sub-combination of these described features.

### BRIEF DESCRIPTION OF DRAWINGS

Embodiments will now be described, by way of example only, with reference to the accompanying drawings which are meant to be exemplary, not limiting, and wherein like elements are numbered alike in several Figures, in which:

FIG. 1A-FIG. 1C illustrates the conventional electrophoresis-based DNA nanopore sensing;

FIG. 2A-FIG. 2C illustrates the diffusiophoresis sensing method according to one embodiment of the present invention;

FIG. 3 illustrates the analyzing apparatus and sensing method according to one embodiment of the present invention;

FIG. 4A-FIG. 4B respectively show the schematic and picture of the cells;

FIG. 5 shows the transmission electron microscopy image of the nanopore and schematic of diffusiophoretic DNA sequencing using a monolayer molybdenum disulfide nanopore;

FIG. 6A shows the experimental results of conventional resistive pulse sensing of ssDNA oligonucleotides using a silicon nitride nanopore;

FIG. 6B-FIG. 6C shows the experimental results of diffusiophoretic sensing of ssDNA oligonucleotides using a monolayer molybdenum disulfide nanopore;

FIG. 7A shows the experimental results of conventional resistive pulse sensing of 1-DNA (dsDNA 48.5 kbp) using a silicon nitride nanopore;

FIG. 7B shows the experimental result obtained by the diffusion current method under a salt concentration gradient; and

FIG. 8 shows the experimental diffusiophoresis sensing results of a designed 60-mer ssDNA molecule (3′-AGAGAGAGAGAGAGAGAGAGAGAGAGAGAGAGAGAGAGAGAGAGAGAG AGAGAGAGAGAG-5′, SEQ ID NO:1) using a monolayer molybdenum disulfide nanopore.

### DETAILED DESCRIPTION

The invention will now be described based on preferred embodiments which do not intend to limit the scope of the present invention but exemplify the invention. All of the features and the combinations thereof described in the embodiment are not necessarily essential to the invention.

### I. Conventional Nanopore Sensing

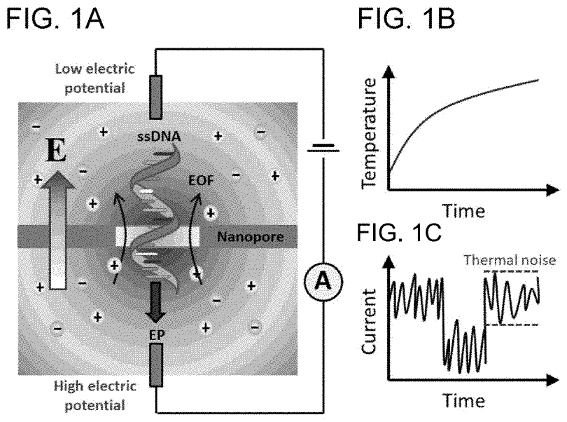

FIG. 1A-FIG. 1C illustrates the conventional resistive pulse DNA nanopore sensing using electrophoresis. In FIG. 1A, E denotes the applied electric field. In the conventional sensing, the conduction current is measured as an external electric potential difference is imposed across the nanopore. When the electric field E is imposed, the temperature within the nanopore can significantly increase due to Joule heating as illustrated in FIG. 1B, resulting in high thermal noise in output current signals as shown in FIG. 1C. In addition, the temperature increase could damage the DNA molecules deteriorating the detection accuracy.

### I. Diffusiophoretic Method

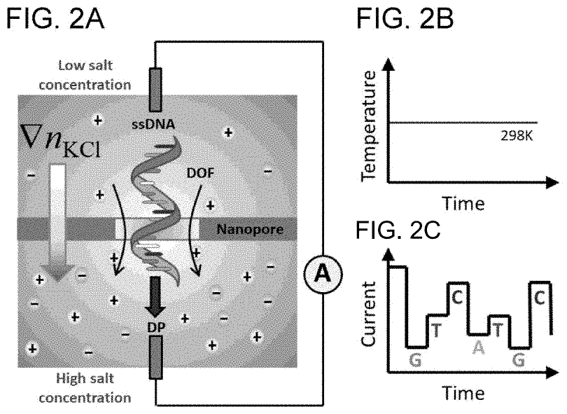

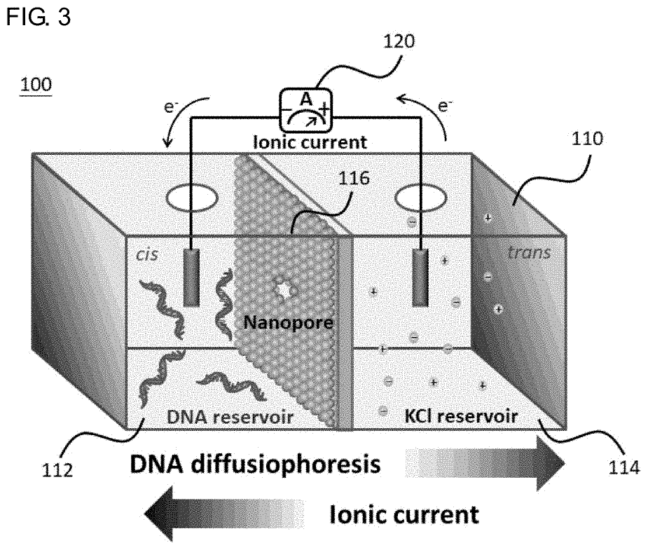

FIG. 2A-FIG. 2C and FIG. 3 illustrate the diffusiophoretic method and an analyzing apparatus according to one embodiment of the present invention, respectively.

As shown in FIG. 3, the analyzing apparatus 100 comprises a device 110, and a current sensor 120. The device 110 has two chambers 112 and 114 (which are called solution cells) which are separated by the nanopore chip 116. Initially, the two solution cells 112 and 114 are filled with different concentration of salinity solutions so as to generate the concentration difference across the nanopore chip 116. In FIG. 2A, ∇n_{KCl} denotes the KCl (potassium chloride) electrolyte concentration gradient.

In case of the nanopore chip 116 is a monolayer MoS₂. The thickness of the layer is 0.65 nm, and the diameter of the nanopore is about a few nanometers (2 nm-4 nm).

As long as the materials' thickness is comparable to the gap between two nucleotides bases (0.34 nm), they could be suitable for high resolution sensing. Therefore, two-dimensional materials such as graphene, graphene oxide, boron nitride (BN), molybdenum disulfide (MoS₂) and tungsten disulfide (WS₂) monolayers are potential candidates for this invention. For thicker materials, silica and silicon nitride can be as thin as 5 nm, which can also be candidates but worse resolution is expected.

Amongst these materials, graphene and MoS₂ have attracted most attention. The reason why MoS₂ is more favorable over graphene lies in the fact that the hydrophobic force between graphene and biomolecules is too strong, making DNA molecules difficult to pass the pore.

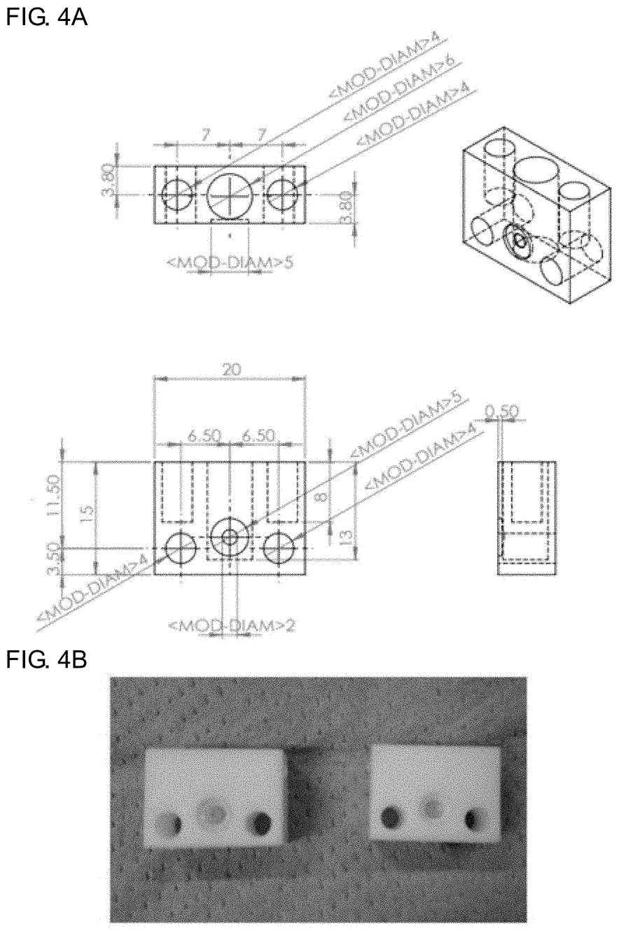

The device 110 has two cells corresponding to the chambers 112 and 114 in FIG. 3. Each cell has an opening in the middle of its top surface, and the solution is poured into the cell via this opening. The electrode is inserted into the solution. Both cells have openings facing each other, and the nanopore chip is sandwiched between the openings of the cells by PDMS (polydimethylsiloxane). FIG. 4A-FIG. 4B respectively show the schematic (by SOLIDWORKS) and picture of the cells.

There are two components in this system: (i) ions (solute) and (ii) water (solvent). When mentioning about the osmotic flow, it refers to the motion of the solvent but not the solute. The transport of solute is governed by the Nernst-Planck equation (if only considering diffusion), whereas the transport of solvent is described by the Navier-Stokes equation that the electric body force term drives the flow.

The current sensor 120 measures the ionic current due to an applied salt concentration difference across a cation selective (negatively charged) two-dimensional monolayer molybdenum disulfide (MoS₂) nanopore. The details of the current sensor 120 is described elsewhere (Non-patent document 1), which comprises a transimpedance amplifier and an A/D converter.

Referring back to FIG. 2A-FIG. 2C, the issues of the conventional method can be avoided by employing a salt concentration gradient, instead of imposing the electric field. In this method, the DNA molecule is detected by ionic current generated by the concentration gradient through a cation selective nanopore, largely suppressing Joule heating effects. Accordingly, the temperature can be kept constant in time as shown in FIG. 2B, and the thermal noise in the detected current is suppressed as shown in FIG. 2C.

Furthermore, in the electric filed-based system that the electroosmotice flow (EOF) is in the opposite direction to the moving electrophoretic (EP) direction of the molecule. In contrast to this, in the salt gradient system, the flow, diffusioosmosis (DOF), direction within the nanopore is the same as the molecule diffusiophoretic (DP) translocation direction. Consequently, the proposed system has a high molecule capture rate and hence high throughput.

The present method is based on a non-equilibrium state process. Considering the ionic flux=(nanopore cross sectional area)×(ionic diffusivity)×(concentration difference)/(nanopore thickness)=4.65×10¹⁰ molecule/s and the total number of cations in the reservoir=(solution volume=1×10⁻⁴ L)×2 M×6.02×10²³=1.2×10²⁰, it is estimated the relaxation time for the process to reach equilibrium is 2.6×10⁹ s=3×10⁴ days. Such a relaxation time is long enough to complete the sensing of DNAs.

The advantages of the new method are:

(i) apart from the nanopore electroosmotic flow that yields excess molecule translocation speed reducing the sensing resolution, the mild diffusioosmotic flow along the nanopore enables much slower molecule translocation speed favorable to molecule detection; and

(ii) The removal of the applied electric field avoids Joule heating enabling isothermal molecule sensing, that not only minimizes the thermal noise but also diminishes the possibilities of molecule thermal damage during sensing and thus high resolution signals can be obtained.

### III. Experimental Processes of the Diffusiophoresis DNA Sequencing Using a Two-dimensional Nanopore

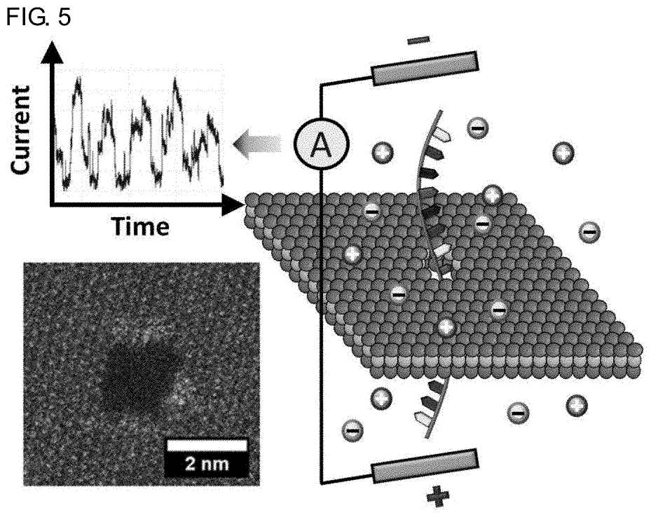

Nanopore ssDNA sequencing experiments were conducted according to the following steps. (I) We first drilled a nanopore (˜500 nm in diameter) using focused ion beam (SMI3050: SII Nanotechnology) on a silicon nitride membrane on top of a Si substrate with a square window of 100 micron at its center. (II) A MoS₂ monolayer layer (˜10 micron×10 micron) was obtained by exfoliation of MoS₂ crystal and mounted onto the silicon nitride pore via PDMS transfer (Non-patent document 6). (III) Following that, a nanopore was sculptured by electron (e-beam) irradiation under transmission electron microscopy (TEM) as shown in FIG. 5. (IV) The two-dimensional nanopore chip was mounted onto Teflon cells and fixed by PDMS. (V) The solution tanks were poured with different concentrations of electrolyte solutions and analytes were added into the cis reservoir. After inserting Ag/AgCl electrodes into each reservoir, an ultra-low noise current measurement system was employed to detect ultra-low noise current signals.

### IV. Results and Discussion

Due to the MoS₂ surface is negatively charged in aqueous solutions, the cation concentration in the nanopore is higher than the bulk solute concentration. In contrast, the chloride ions are repelled from the surface resulting a partially cation selective membrane. Regarding the conventional resistive pulse sensing system where an electric potential difference is applied on the electrodes between the reservoirs, the external electric field drives negatively charged DNA molecules toward the trans reservoir possessing a higher electric potential (i.e. electrophoresis). In contrast, due to the negatively charged surface, the positively charged solution in the nanopore flows to the cis reservoir. In case of diffusiophretic transport that a concentration exists between two reservoirs, the nonuniform concentration in the axial direction in the nanopore drives negatively charged DNA molecules toward the high concentration end due to polarization effects of the electric double layer (Non-patent document 7).

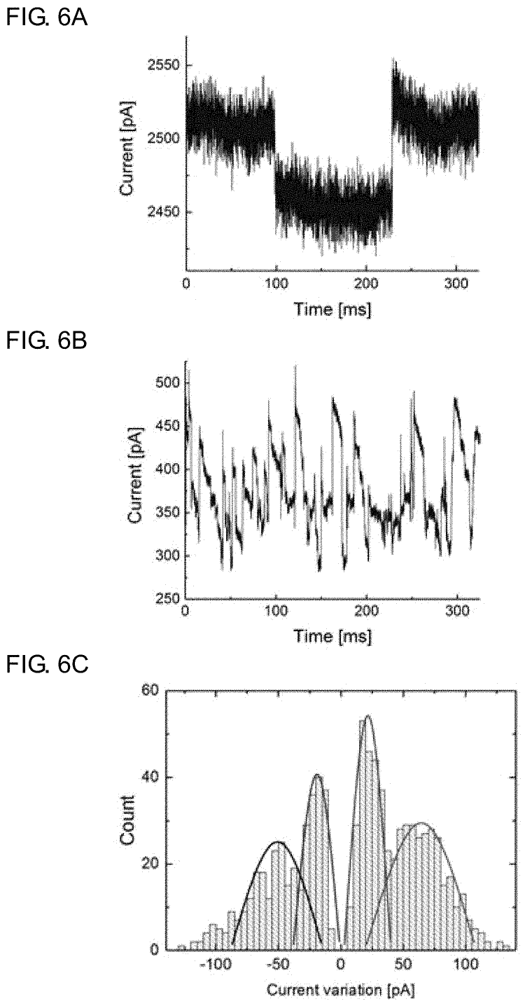

To compare with results of conventional resistive pulse sensing methods, we conducted a control experiment. FIG. 6A shows a typical current variation signal of conventional conduction current-based sensing using a 20 nm thick silicon nitride nanopore immersed in a 1M potassium chloride electrolyte solution. An overall translocation signal was shown and the detailed structural information was hidden due to the huge thermal noise and fast translocation time. This result is consistent with the present literature.

On the other hand, as shown in FIG. 6B, the structural information of ssDNA Oligonucleotides can be revealed using a solid-state nanopore (first time in history), due to the slowdown of the molecules and elimination of Joule heating effects. The proposed diffusiophoretic sensing method for ionic current measurements, as molecules migrate from a low solute concentration reservoir (0.01M potassium chloride electrolyte solution) to a high solute concentration reservoir (2M potassium chloride electrolyte solution) via an two-dimensional monolayer molybdenum disulfide nanopore (approximately 3.5 nm in diameter), revealed clear structural information of the detected oligonucleotide.

The histogram of the current measurement system in FIG. 6C indicates four peaks of current variation levels, representing different types of nucleotides on the ssDNA molecule. Note that, even for the commercialized nanopore sequencer MinION by Oxford Nanopore Technologies using biological nanopores, it is difficult to direct read out the sequence by eye without further analysis. Normally machine learning is engaged to decipher these signals. However, it is clear that the invented method is powerful to provide high resolution of molecule structure using solid-state nanopore which cannot achieve by other methods.

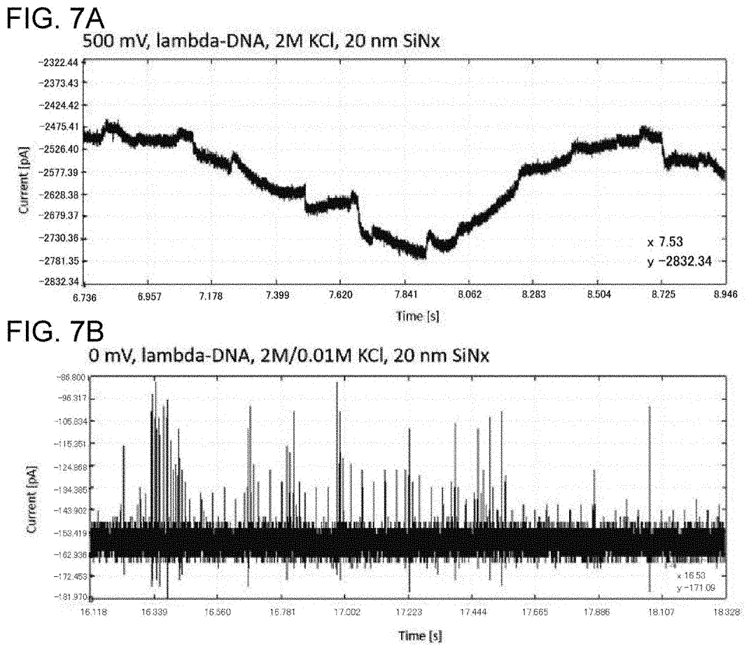

It is to be noted that, the diffusiophoresis method is not limited to ultrathin nanopores and it can be applied to thicker nanopores. FIGS. 7A and 7B shows the experimental results with a 20 nm Silicon nitride nanopore. FIG. 7A shows the blockage signal of 1-DNA (dsDNA 48.5 kbp) obtained by the conventional resistive pulse sensing method under an electric field, and FIG. 7B shows that obtained by the diffusion current method under a salt concentration gradient. Observed advantages of the diffusion current method over the conventional conduction current method are:

Higher signal frequency (more peaks at the same recording period);

Higher signal to noise ratio; and

Distinguishable peak magnitudes.

In the test with the 20 nm Silicon nitride nanopore, the single nucleotides identification should be difficult for both cases due to the limitation of spatial resolution (20 nm>>0.3 nm of the gap between each nucleotide pair). However, it is still clear that the diffusiophoresis method achieves higher resolution over the same time period and the noise magnitude was about 50% smaller (about 30 pA versus 15 pA).

Nevertheless, it is expected the ionic current decreases with increase of the pore length, which could raise the current measurement difficulty. So, for thicker pores, larger pore diameters are needed, which can be used to detect the structure of larger molecules (such as proteins). Otherwise, femto ampere (fA) current measurement systems have to be used.

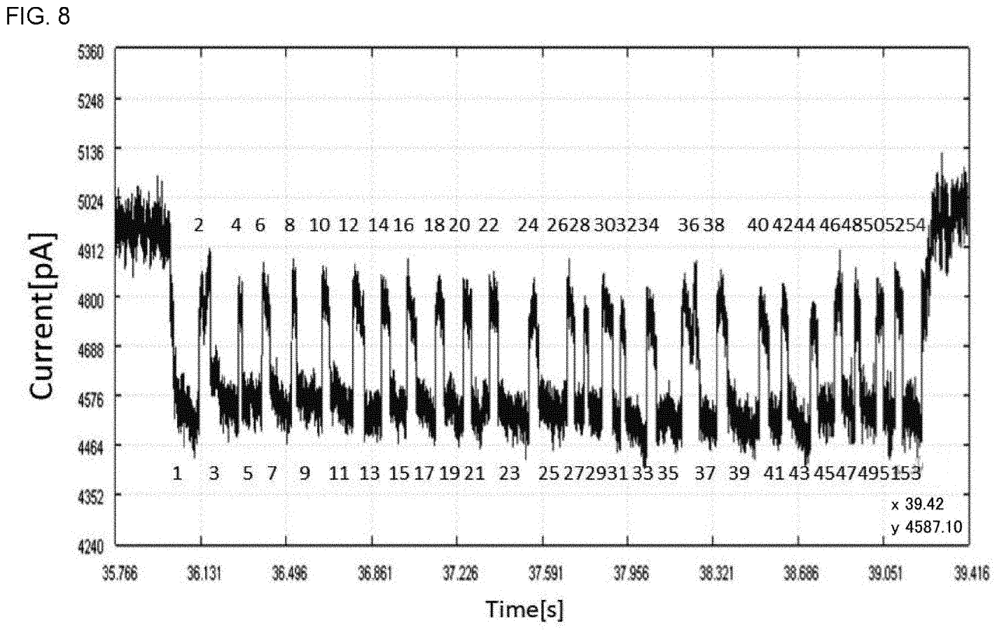

Similar to the current signals from biological nanopores, the current variation does not only depend on the nucleotide types, but can be affected by the sequence of the nucleotides and secondary structure of ssDNA recombination, preventing the direct readout of the sequence without advanced post analysis (e.g. via machine learning). In this regard, we evaluate the accuracy of the diffusiophoretic DNA sequencing method utilizing a designed ssDNA 60-mer containing only two kinds of nucleotides, Adenosine triphosphate (A) and Guanosine triphosphate (G) to prevent secondary structure.(3′-AGAGAGAGAGAGAGAGAGAGAGAGAGAGAGAGAGAGAGAGAGA GAGAGAGAGAGAGAGAG-5′, SEQ ID NO:1) The experimental results showed a repeated current patent as seen in FIG. 8. Note that, we obtained this periodic current signal only when testing this designed molecule and the detected peak numbers (54) is close to the base number of the designed molecule. For other ssDNA molecules with four nucleotide species, multi-level signals appeared.

### V. Conclusion

We propose a novel and simple approach to effectively resolve above issues originated from the external electric field. Instead of tracing the conduction current variation, we replaced the external electric field with a solute concentration difference across a monolayer molybdenum disulfide nanopore and detected the ionic current variation during the diffusiophoretic translocation events of ssDNA oligonucleotides. In this system, Joule heat effects were avoided. As a result, we successfully obtained structural signals of nucleotides on the DNA molecules. These promising results revive opportunities for direct DNA sequencing using solid-state nanopores.

### VI. Application

The application of the present invention is not limited to the DNA sequencer. The present invention is useful in various applications, such as life cell analysis or large molecular analysis etc.

Nanopore technology has emerged as a revolutionary technique replacing conventional sequencing methods that require a considerable amount of time and money. Currently this nanopore sequencing market is dominated by Oxford Nanopore Technologies utilizing biological nanopores for molecule sensing. Although it has been craved for long time to use solid-state materials for molecule sequencing which are expected to be more competitive than biological nanopores in terms of robustness and reliability, no successful structural results had been reported in the past two decades since the idea was envisaged. Therefore, this very first method showing clear structural ssDNA oligonucleotides information will have huge impact on the present nanopore technology market. It is not difficult to predict that within a few years the molecule sequencing using solid-state nanopores will be dominant over biological nanopores in the global market. Undoubtedly, the potential of the invention is enormous for commercial purposes.

What is claimed is:

1. An analyzing apparatus comprising:
a pore device having a cation selective nanopore, and a first chamber and a second chamber which are separated by the cation selective nanopore, wherein in an initial state, the first chamber includes molecules to be analyzed, and the second chamber has higher salt concentration than the first chamber;
a first electrode provided in the first chamber;
a second electrode provided in the second chamber; and
a current sensor structured to measure an ionic current flowing through the first electrode and the second electrode.

2. The analyzing apparatus according to claim 1, wherein a material of the cation selective nanopore is one of graphene, graphene oxide, boron nitride (BN), molybdenum disulfide (MoS₂) and tungsten disulfide (WS₂).

3. The analyzing apparatus according to claim 1, wherein each of the first chamber and the second chamber has an opening at its top surface, through which the first electrode and the second electrode are inserted.

4. The analyzing apparatus according to claim 2, wherein each of the first chamber and the second chamber has an opening at its top surface, through which the first electrode and the second electrode are inserted.

5. An analyzing method comprising:
providing a pore device having a cation selective nanopore and a first chamber and a second chamber which are separated by the cation selective nanopore;
pouring solution into the first chamber and the second chamber, wherein the second chamber has higher salt concentration than the first chamber;
providing molecules to be analyzed in the first chamber; and
measuring an ionic current flowing through a first electrode and a second electrode which are respectively provided in the first chamber and the second chamber.
